(19) United States
(12) Patent Application Publication
(10) Pub. No.: US 2024/0218373 A1
(43) Pub. Date: Jul. 4, 2024

(54) METHODS AND COMPOSITIONS FOR TREATING NEUROINFLAMMATION

(71) Applicant: AIM ImmunoTech Inc., Ocala, FL (US)

(72) Inventors: Thomas K. EQUELS, Ocala, FL (US); David R. STRAYER, Bryn Mawr, PA (US)

(73) Assignee: AIM ImmunoTech Inc., Ocala, FL (US)

(21) Appl. No.: 18/558,498

(22) PCT Filed: May 5, 2022
(86) PCT No.: PCT/US2022/027942

§ 371 Date: Nov. 1, 2023

Related U.S. Application Data
(60) Provisional application No. 63/197,903, filed on Jun. 7, 2021.
(60) Provisional application No. 63/184,752, filed on May 5, 2021.

(51) Int. Cl. C12N 15/113; A61P 25/28

(52) U.S. Cl. CPC C12N 15/1138; A61P 25/28

(57) ABSTRACT

Methods and compositions for treating, reducing, or preventing neuroinflammation in a subject in need thereof. The methods comprise, at least, a step of administering to a subject a therapeutically effective amount of Therapeutic Double-Stranded RNA. The compositions comprise at least a therapeutically effective amount of Therapeutic Double-Stranded RNA.

### PRIORITY

This application claims the benefit of priority to U.S. Provisional Patent Application No. 63/184,752 filed May 5, 2021, and 63/197,903 filed Jun. 7, 2021, the entire contents of which are incorporated herein by reference.

### BACKGROUND

Neuroinflammation is a possible pathological contributor to many neurodegenerative diseases. It may be possible that neuroinflammation, usually including activation of glial cells, such as microglia and astrocytes, and release of cytotoxic compounds, e.g., cytokines and reactive oxygen species (ROS), are able to cause neuronal damage and death. Therefore, there is an unmet need for a successful therapy for neuroinflammation.

### BRIEF DESCRIPTION

One embodiment is directed to a method for treating, reducing, or preventing neuroinflammation in a subject in need thereof, comprising: administering to the subject a composition comprising a therapeutically effective amount of tdsRNA; wherein the tdsRNA is at least one selected from the group consisting of:

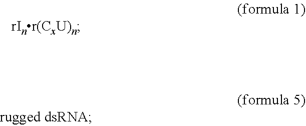

wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

In any embodiment of this disclosure, including, for example, in the method and the composition of the disclosure, the neuroinflammation may be at least one selected from the group consisting of: a neuroinflammation disorder; a neuroinflammation disorder symptom; a neuroinflammation disorder pathology; and a neuroinflammation disorder sign. A neuroinflammation disorder sign may be a phenotype that is correlated with a neuroinflammation disorder such as a high blood level of a marker such as amyloid accumulation and others discussed in this disclosure.

In any embodiment of this disclosure, the neuroinflammation may be selected from the group consisting of: neuroinflammation; chronic neuroinflammation; depression; schizophrenia; Alzheimer's disease (AD); Parkinson's disease; multiple sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); multi-system atrophy; acute disseminated encephalomyelitis (ADEM); acute optic neuritis (AON); transverse myelitis; and Neuromyelitis Optica (NMO).

In any embodiment, the method and composition may reduce, slow, reverse or prevent an increase of the Cognitive Deficit (CD) subscale of the SCL-90-R Scale in a subject. An increase of the Cognitive Deficit (CD) subscale of the SCL-90-R Scale indicates more disability and more CD. Therefore, reducing, slowing, reversing or preventing an increase, as discussed above, is beneficial to the subject.

In any of the methods, the method may further comprise a step of determining that the subject has or is at risk of developing: (1) a neuroinflammation disorder; (2) a neuroinflammation disorder symptom; (3) a neuroinflammation disorder pathology; or (4) a neuroinflammation disorder sign; before the administering step.

In any embodiment, the disorder symptom, disorder pathology, or disorder sign may be at least one selected from the group consisting of: amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; an increasing Cognitive Deficit (CD) subscale of the SCL-90-R Scale; microglial activation; astrocyte activation; elevated IL-6 (interleukin-6), elevated IL-23 (interleukin-23), elevated IL-1B (interleukin-1 beta), elevated TNF-α (tumor necrosis factor alpha), elevated Iba1 (microglial activation), elevated GFAP (astrocytic response), depressed NeuN (neuronal loss), depressed TGF-beta (Transforming growth factor beta), elevated Interferon-γ (IFN-γ), or elevated inducible Nitric Oxide Synthase (iNOS).

In any embodiment, the Cognitive Deficit (CD) subscale of the SCL-90-R Scale comprises the symptoms of headaches; trouble remembering; temper outburst; doing things slow; double check; mind goes blank; trouble remembering; and wrong with body. The subscale measures the amount of cognitive deficit (CD) and, therefore, a person with a higher cognitive deficit subscale score is more impaired. The greater the CD, the greater the score. An unaffected subject would have a score of zero indicating the least CD.

In any embodiment, the tdsRNA may exert its effect by modulating an immune system in the subject by binding a Toll-like receptor 3 (TLR3) receptor in the subject.

In any embodiment, the tdsRNA may function by crossing the blood-brain barrier in the subject.

In any embodiment, the method may reduce, stop or reverse at least one selected from the group consisting of: a neuroinflammatory disorder symptom; a neuroinflammatory disorder pathology; and a neuroinflammatory disorder sign; in the subject.

In any embodiment, the chemical formula term “n” may be a number with a value which is at least one selected from the group consisting of: 40 to 50,000; 40 to 40,000; 50 to 10,000; 60 to 9000; 70 to 8000; 80 to 7000; and 380 to 450.

In any embodiment, “n” in the formula for tdsRNA may be from 40 to 40,000; the tdsRNA may have about 4 to about 4000 helical turns of duplexed RNA strands; or the tdsRNA may have a molecular weight selected from the group consisting of: 2 kDa to 30,000 kDa; 25 kDa to 2500 kDa; and 250 kDa to 320 kDa.

In any embodiment, tdsRNA may comprise rIₙ•ribo(C₁₁₋₁₄U)ₙ; and rugged dsRNA.

In any embodiment, the rugged dsRNA may have a single strand comprised of r(C₄₋₂₉U)ₙ, r(C₁₁₋₁₄U)ₙ, or r(C₁₂U)ₙ; and an opposite strand comprised of r(I); wherein the single strand and the opposite strand do not base pair the position of the uracil base, and wherein the single strand and the opposite strand are partially hybridized.

In any embodiment, the rugged dsRNA may (1) have a molecular weight of about 250 kDa to 500 kDa; (2) each strand of the rugged dsRNA is from about 400 to 800 basepairs in length; or (3) the rugged tdsRNA has about 30 to 100 or 30-60 helical turns of duplexed RNA.

In any embodiment, the tdsRNA may be or may comprise Rugged dsRNA which is resistant to denaturation under conditions that are able to separate hybridized poly(riboinosinic acid) and poly(ribocytosinic acid) strands (rIₙ•rCₙ).

In any embodiment, the rugged dsRNA may be an isolated double-stranded ribonucleic acid (dsRNA) enzymatically active under thermal stress comprising the following properties. Each strand of RNA may have a molecular weight of about 250 KDa to about 500 KDa, 400-800 basepairs, or 30 to 60 helical turns of duplex RNA. The double strand may comprise a single strand comprised of poly(ribocytosinic₄₋₂₉ uracilic acid) and an opposite strand comprised of poly(riboinosinic acid). The two strands do not base pair the position of the uracil base but the two strands base pair the position of the cytosine base, and therefore the two strands are partially hybridized.

In any embodiment, the composition, including a composition used in the method, may comprise at least one pharmaceutically acceptable carrier.

In any embodiment, administering may be one of more of the following: systemic administration; intravenous administration; intradermal administration; subcutaneous administration; intramuscular administration; intranasal administration (e.g., and including pulmonary airway administration); intranasal administration and oral administration; intraperitoneal administration; intracranial administration; intravesical administration; oral administration (e.g., and including through the mouth and/or by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; and dry powder inhalants administration.

In any embodiment, tdsRNA may be administered at one of the following dosages of about: 25 mg to 700 mg of tdsRNA per day; 20 mg to 200 mg of tdsRNA per day; 50 mg to 150 mg of tdsRNA per day; or 80 mg to 140 mg of tdsRNA per day.

In any embodiment, the administration may be performed at a rate selected from the group consisting of: one dose per day; one dose every 2 days; one dose every 3 days; one dose every 4 days; one dose every 5 days; one dose a week; two doses a week; three doses a week; one dose every two weeks; one dose every 3 weeks; one dose every 4 weeks; and one dose a month.

Another embodiment is directed to a composition. The composition may be used for any of the methods of this disclosure and the composition may be used for treating reducing, or preventing neuroinflammation in a subject. The composition comprises

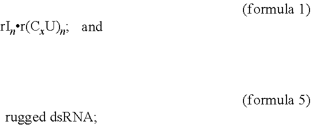

wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

In any embodiment, the neuroinflammation may be at least one selected from the group consisting of a neuroinflammation disorder; a neuroinflammation disorder symptom; a neuroinflammation disorder pathology; a neuroinflammation disorder sign; neuroinflammation; depression; schizophrenia; Alzheimer's disease (AD); Parkinson's disease; Multiple Sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); multi-system atrophy; Acute disseminated encephalomyelitis (ADEM); Acute Optic Neuritis (AON); Transverse Myelitis; and Neuromyelitis Optica (NMO).

In any embodiment, the disorder symptom, disorder pathology, or disorder sign may be at least one selected from the group consisting of amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; an increasing or high Cognitive Deficit (CD) subscale of the SCL-90-R scale (meaning high cognitive deficit and more impaired compared to a normal healthy subject); microglial activation; astrocyte activation; elevated IL-6 (interleukin-6), elevated IL-23 (interleukin-23), elevated IL-1β (interleukin-1 beta), elevated TNF-α (tumor necrosis factor alpha), elevated Iba1 (microglial activation), elevated GFAP (astrocytic response), depressed NeuN (neuronal loss), depressed TGF-beta (Transforming growth factor beta), elevated Interferon-γ (IFN-γ), or elevated inducible Nitric Oxide Synthase (iNOS).

In any embodiment, at least 90 weight percent (wt %) of the tdsRNA may be larger than a size selected from the group consisting of: 40 basepairs; 50 basepairs; 60 basepairs; 70 basepairs; 80 basepairs; and 380 basepairs.

In any embodiment, at least 90 wt % of the tdsRNA may be smaller than a size selected from the group consisting of: 50,000 basepairs; 10,000 basepairs; 9000 basepairs; 8000 basepairs; 7000 basepairs; and 450 basepairs.

In any embodiment, the tdsRNA may comprise 0.1-12 mol % rugged dsRNA; preferably the tdsRNA comprises 0.1-5 mol % rugged dsRNA.

In any embodiment, the tdsRNA may be complexed with a stabilizing polymer. the stabilizing polymer may be at least one selected from the group consisting of: (1) polylysine; (2) polylysine and carboxymethylcellulose; (3) polyarginine; and (4) polyarginine and carboxymethylcellulose.

In any embodiment, the administration may be by intranasal administration and intranasal administration may be at least one selected from the group consisting of: administering to nasal passages; administering to nasal epithelium; administering to lung; administering by inhalation; administering to the larynx; administering to bronchi; administering to alveoli; administering by inhalation; administering by nasal instillation; and administering to the cerebrospinal fluid.

In any embodiment, administering may be administered to at least one tissue or cell (including groups of cells) selected from the group consisting of: airway tissue; nose tissue; oral tissue; alveoli tissue; pharynx tissue; trachea tissue; bronchi tissue; carina tissue; bronchi tissue; bronchioles tissue; lung tissue; lobe of a lung tissue; alveoli tissue; nasal passage tissue; nasal epithelium tissue; larynx tissue; bronchi tissue; inhalation tissue; an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; and a sinus cell.

In any embodiment, administering may be performed by a delivery system comprising the tdsRNA. The delivery system may be at least one selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered dose inhaler; a dry powder inhalation device; an instillation device; an intranasal instillation device; an intravesical instillation device; a swab; a pipette; a nasal irrigation device; a nasal rinse; an aerosol device; a metered aerosol device; a pressurized dosage device; a powdered aerosol device; a spray aerosol device; a spray device; a metered spray device; and a suspension spray device.

In any embodiment, administering may be administering tdsRNA to the subject to increase tdsRNA levels in the cerebrospinal fluid of the subject. In any embodiment, administering may be (1) direct administering to the cerebrospinal fluid of the subject. In any embodiment, administering may be by administering a tdsRNA to the subject wherein the tdsRNA crosses a blood-brain barrier in the subject and increases tdsRNA levels in the cerebrospinal fluid of the subject.

Another embodiment is directed to a method for treating, reducing, or preventing (1) a neuroinflammation disorder/disease, (2) a neuroinflammation disorder/disease symptom, (3) a neuroinflammation disorder/disease pathology, or (4) a neuroinflammation disorder/disease sign, in a subject in need thereof. The method comprises the step of administering to the subject a composition comprising a therapeutically effective amount of therapeutic double-stranded RNA (tdsRNA). The tdsRNA in any embodiment of the disclosure is at least one selected from the group consisting of: rIₙ•r(CₓU)ₙ (formula 1); rIₙ•r(CₓG)ₙ (formula 2); rAₙ•rUₙ (formula 3); rIₙ•rCₙ (formula 4); and rugged dsRNA (formula 5); wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35. Another embodiment is directed to a composition comprising the tdsRNA.

Examples of various embodiments of this disclosure are provided. Each part or subpart of an embodiment should also be considered an embodiment. Each of the embodiments of this disclosure may be combined with one or more other embodiments of the disclosure and the combination is also an embodiment of this disclosure. In any part of this disclosure and in any embodiment, the terms “comprise,” “comprising,” “consist,” “consisting,” “consist essentially of,” or “consisting essentially of” can be replaced with each other and each of these replacements is also an embodiment of the disclosure. That is, the interchange of one of the above-listed terms of this paragraph with another, even if they have different meanings, is also envisioned and is an embodiment. In this disclosure, the term “disease” may be substituted with the term “disorder.” and the term “disorder” may be substituted with the term “disease.” Various aspect of the above disclosure is discussed in more detail below.

### DETAILED DESCRIPTION

### 1. Definitions

This disclosure relates to, inter alia, tdsRNA. tdsRNA can also be called “therapeutic dsRNA,” or “therapeutic double-stranded RNA” and these terms have the same meaning.

“r” and “ribo” have the same meaning and refer to ribonucleic acid or the nucleotides or nucleosides that are the building block of ribonucleic acid.

RNA consists of a chain of linked units called nucleotides. This disclosure relates mostly to RNA and, therefore, unless otherwise specified, the nucleotides and bases expressed refers to the ribo form of the nucleotide or base (i.e., ribonucleotide with one or more phosphate groups). Therefore “A” refers to rA or adenine, “U” refers to rU or uracil, “C” refers to rC or cytosine, “G” refers to rG or guanine, “I” refers to rI or inosine, “rN” refers to rA, rU, rC, rG or rI. Each of these (i.e., A, U, C, G, I) may have one or more phosphate groups as discussed above depending on whether they are part of a chain (i.e., RNA) or free (nucleoside, nucleotide, etc.).

“n” is a positive number and refers to the length of a ssRNA or dsRNA or to the average length of a population of ssRNA or dsRNA. “n” can be a positive integer when referring to one nucleic acid molecule or it can be any positive number when it is an average length of a population of nucleic acid molecules.

An RNA may have a ratio of nucleotides or bases. For example, r(C₁₂U)ₙ denotes a single RNA strand that has, on average 12 C bases or nucleotides for every U base or nucleotide. As another example, r(C₁₁₋₁₄U)ₙ denotes a single RNA strand that has, on average 12 C bases or nucleotides for every U base or nucleotide.

Formulas: As an example, the formula “rIₙ•r(C₁₂U)ₙ” can be expressed as riboIₙ•ribo(C₁₂U)ₙ, rIₙ•ribo(C₁₂U)ₙ, or riboIₙ•r(C₁₂U)ₙ, refers to a double-stranded RNA with two strands. One strand (rIₙ) is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U). The terms “r” and “ribo” have the same meaning in the formulas of the disclosure. Thus, rI, riboI, r(I) and ribo(I) refer to the same chemical which is the ribose form of inosine. Similarly, rC, riboC, r(C) and ribo(C) all refer to cytidine in the ribose form which is a building block of RNA. rU, riboU, r(U) and ribo(U) all refer to Uracil in the ribose form which is a building block of RNA.

The “•” symbol indicates that one strand of the dsRNA is hybridized (hydrogen-bonded) to the second strand of the same dsRNA. Therefore, rIₙ•r(C₁₂U)ₙ is double-stranded RNA comprising two ssRNA. One ssRNA is poly(I) and the other ssRNA is poly(C₁₂U). It should be noted that while we referred to the two strands being hybridized, not 100% of the bases form base pairing as there are some bases that are mismatched. Also, because rU does not form base pairing with rI as well as IC form base paring with rI, rU provides a focus of hydrodynamic instability in rIₙ•r(C₁₂U)ₙ at the locations of the U bases.

As another example, the formula “rIₙ•r(C₁₁₋₁₄U)ₙ” refers to the same dsRNA except that a ratio of C bases to U bases one strand is about 11 to about 14. That is, the ratio can be 11, 12, 13 or 14 or any value between 11 and 14. For example, when half of the strands are r(C₁₂U)ₙ and half of the strands are r(C₁₃U)ₙ, the formula would be r(C_{12.5}U)ₙ.

The dsRNA (tdsRNA) and ssRNA of this disclosure are homopolymers (e.g., a single-stranded RNA where every base is the same) or heteropolymers (e.g., a single-stranded RNA where the bases can be different) of limited base composition. The tdsRNAs are not mRNA and are distinct from mRNA in structure. For example, the ssRNA and dsRNA are preferably missing one or all of the following: (1) 5′ cap addition, (2) polyadenylation, (3) start codon, (4) stop codon, heterogeneous protein-coding sequences, and (5) spice signals.

### 2. Neuroinflammatory Disorders

Neuroinflammation or neuroinflammatory disease/disorder refers to a process or processes whereby the brain's immune system is triggered following an inflammatory challenge such as those posed by injury, infection, exposure to a toxin, neurodegenerative disease, or aging. In these cases, the central nervous system (CNS) is activated to mount an immune response.

When there is a disease or disorder, it is believed that the microglia upregulate inflammatory signals, resulting in chronic neuroinflammation which leads to a chronic disorder or disease. Sometimes, the inflammatory response leads to the secretion of biomarkers (including cytokines). Abnormal levels of these biomarkers, such as elevated levels or low levels relative to normal, indicate neuroinflammation. The markers may be in blood, cerebralspinal fluid, or other body fluids. Examples of such biomarkers include elevated IL-6 (interleukin-6), elevated IL-23 (interleukin-23), elevated IL-1β (interleukin-1 beta), elevated TNF-α (tumor necrosis factor alpha), elevated Iba1 (microglial activation), elevated GFAP (astrocytic response), depressed NeuN (neuronal loss), depressed TGF-beta (Transforming growth factor beta), elevated Interferon-γ (IFN-γ), or elevated inducible Nitric Oxide Synthase (iNOS).

“Elevated,” “increased,” or “high” refers to an elevated level, or increased level, or a high level as compared to the level of a person, preferably age and sex-matched, that does not suffer from neuroinflammation or any neuroinflammation disease. Similarly, “depressed,” “decreased,” or “low” refers to a depressed level, or decreased level, or a low level as compared to the level of a person, preferably age and sex-matched, that does not suffer from neuroinflammation or any neuroinflammation disease.

Other signs of neuroinflammation or neuroinflammatory disease/disorder include: amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; a high or increasing score on a Cognitive Deficit (CD) subscale of the SCL-90-R Scale; microglial activation; or astrocyte activation. Further neuroinflammation or neuroinflammatory disease/disorder may manifest as a disease or disorder symptom such as the one or more symptoms of: neuroinflammation; depression; schizophrenia; Alzheimer's disease (AD); Parkinson's disease; Multiple Sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); multi-system atrophy; Acute disseminated encephalomyelitis (ADEM); Acute Optic Neuritis (AON); Transverse Myelitis; or Neuromyelitis Optica (NMO). One preferred method of detecting neuroinflammation is a high or increasing Cognitive Deficit (CD) subscale of the SCL-90-R Scale (indicating high or increasing CD).

Other markers or symptoms for neuroinflammation, which can be detected individually, together, or as a ratio, include, for example, amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; a high or increasing score on a Cognitive Deficit (CD) subscale of the SCL-90-R scale; microglial activation; astrocyte activation; elevated IL-6 (interleukin-6), elevated IL-23 (interleukin-23), elevated IL-1 (interleukin-1 beta), elevated TNF-α (tumor necrosis factor alpha), elevated Iba1 (microglial activation), elevated GFAP (astrocytic response), depressed NeuN (neuronal loss), depressed TGF-beta (Transforming growth factor beta), elevated Interferon-γ (IFN-γ), or elevated inducible Nitric Oxide Synthase (iNOS).

Neuroinflammatory diseases, such as Alzheimer's disease (AD), were once a clinical diagnosis confirmed by postmortem autopsy. Today, with the development of many diagnostic methods for neuroinflammatory biomarkers, laboratory assays to detect neuroinflammatory diseases are able to detect and confirm clinical diagnosis in symptomatic individuals. A variety of assays, such as assays for Alzheimer's disease, are commercially available.

Listed below is a brief summary of the tests available for a representative neuroinflammatory disease—Alzheimer's disease. For Alzheimer's disease, cerebrospinal Fluid Aβ42 peptide (also referred to as Ab42), Aβ40 peptide (also referred to as Ab40) and tau protein are common biomarkers and their presence or ratio can be measured by either mass spectrometry or immunoassay. In the future, these assays may be extended to measure blood biomarkers. These assays are performed commercially by Athena Diagnostics and Quest, which measure CSF Aβ42 and Aβ40 as laboratory-developed tests. Both Roche Diagnostics and Fujirebio have developed immunoassays for Aβ42 and Aβ40 and both manufacturers received breakthrough device designation to accelerate Food and Drug Administration (FDA) clearance for their automated platforms.

For example, listed below are the biomarker|Testing Method|AD pathology threshold|Healthy Control levels (if available)|Manufacturer for a number of Alzheimer's disease tests. In this summary paragraph, a “/” refers to a ratio. Thus, Ab42/Ab40 refers to a ratio of Ab42 to Ab40 expressed as a quotient. Similarly, Ab42/t-tau is the ratio of Ab42 to t-tau. Ab42 in CSFIELISA|570 pg/mL|1124 pg/mLb|Fujirebio INNOTEST; Ab42 in CSF|Luminex xMAPINA|Fujirebio INNO-BIA; Ab42 in CSF|CLEIA|<916 pg/mL|Fujirebio Lumipulse; Ab42 in CSF|ECL|<1098 pg/mL|Roche Elecsys; Ab42 in CSF|ELISA|<507.5 pg/mL|Euroimmun; Ab42 in CSF|ECLIA|<495.9|MSD; Ab42 in CSF|Simoa|<1742 pg/mL|Quanterix; Ab42 in CSF|SPE+LC-MS/MSI511 pg/mL|1152 pg/mL|Reference Method-C12RMP1; Ab42 in CSF|Isotope Dilution Mass specl<1059 pg/mL|Reference Method-C11RMP9; Ab42/Ab40, CSF|ECLIA|<0.075|Roche Elecsys; Ab42/Ab40, CSF|CLEIA|0.062|Fujirebio Lumipulse; Ab42/Ab40, CSF|ELISA|<0.10|Euroimmun; Ab42/Ab40, CSF|ECLIA|<0.09|MSD; Ab42/Ab40, CSF|Simoa|<0.16|Quanterix; Ab42/Ab40, CSF|Isotope Dilution Mass spectrometryk|<0.085|Reference Method-C11RMP9; T-tau, CSF|ELISA|290 pg/mL|635 pg/mLb|Fujirebio INNOTEST; T-tau, CSF|Luminex xMAP|NA|Fujirebio INNO-BIA; T-tau, CSF|CLEIA|>456 pg/mL|Fujirebio Lumipulse; T-tau, CSF|ECLIA|>242 pg/mL|Roche Elecsys; T-tau, CSF|ELISA|>412 pg/mL|Euroimmun; T-tau, CSF|Isotope Dilution Mass spec|29 pmol/L|17 pmol/L|Merck; P-tau, CSF|ELISA|48 pg/mL|79 pg/mL|Fujirebio INNOTEST; P-tau, CSF|Luminex xMAP|NA|Fujirebio INNO-BIA; P-tau, CSF|CLEIA|>63 pg/mL|Fujirebio Lumipulse; P-tau, CSF|ECLIA|>19.2 pg/mL|Roche Elecsys; P-tau/Ab42, CSF|ELISA|NA|Fujirebio INNOTEST; P-tau/Ab42, CSF|Luminex xMAP|NA|Fujirebio INNO-BIA; P-tau/Ab42, CSF|CLEIA|>0.068|Fujirebio Lumipulse; P-tau/Ab42, CSF|ECLIA|>0.0198|Roche Elecsys; Ab42/tau|ELISA|<1.47|Euroimmun; and Ab42/t-tau and pTau, CSF|ELISA|ATI<1, pTau>61 pg/mL|Admark.

### 3. Therapeutic Double-Stranded RNA

The double-stranded RNAs described in this disclosure are therapeutic double-stranded RNA, abbreviated as “tdsRNA.” tdsRNA includes, at least, Rintatolimod which is a tdsRNA of the formula rIₙ•r(C₁₂U)ₙ). tdsRNA may be stored or administered in a pharmaceutically acceptable solution such as Phosphate Buffered Saline (PBS).

The tdsRNA may be a tdsRNA produced by any of the methods of this disclosure—referred to herein as the “tdsRNA Product” or “tdsRNA”—the two terms have the same meaning. tdsRNA can be represented by one or more of the formulas below in any combination:

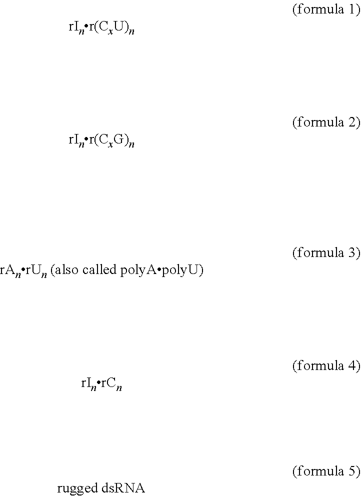

Each will be discussed further below.

The tdsRNA may be represented by one or more of the formulas as follows:

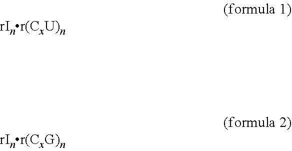

x may be at least one selected from the group consisting of: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29 (4 to 29), 4-30 (4 to 30), 4-35 (4 to 35), 11-14 (11 to 14), 30-35 (30 to 35). Of these, x=12, and x=11-14 (x may be any value between 11 to 14) are especially preferred.

In these formulas 1 to 5, and in other formulas, where there is no subscript next to a base, the default value is “1.” For example, in the formula rIₙ•r(C₁₂U)ₙ, there is no subscript following “U,” it is understood that rIₙ•r(C₁₂U), is the same as rIₙ•r(C₁₂U₁)ₙ and the formula is meant to convey that for the strand denoted as r(C₁₂U₁)ₙ, there are 12 rC base for every rU base. Thus, x is also a ratio of the bases of one strand of the tdsRNA. The length of the tdsRNA strand is denoted as a lowercase “n” (e.g., rIₙ•r(C₁₂U)ₙ). The subscript n is also the length of each individual single-stranded nucleic acid. Since tdsRNA is double-stranded, n is also the length of the double-stranded nucleic acid—i.e., the length of the tdsRNA. For example, rIₙ•r(C₁₂U)ₙ indicates, inter alia, a double-stranded RNA with each strand with a length of n.

In another aspect, the tdsRNA may have a formula as follows:

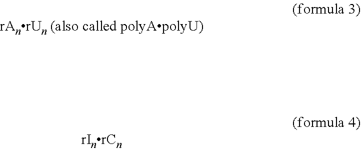

In another aspect, the tdsRNA may be a rugged dsRNA (formula 5).

In one embodiment, tdsRNA is one or more at least one selected from the group consisting of formula 1, formula 2, formula 3, formula 4, and formula 5. In another embodiment, tdsRNA comprises formula 1 and formula 2 only. In one preferred embodiment, tdsRNA comprises formula 1 only. In another embodiment, tdsRNA comprises formula 1 and formula 5 (rugged dsRNA) only.

In another aspect, at least 70%, at least 80%, or at least 90% of the tdsRNA may have a molecular weight of between 400,000 Daltons to 2,500,000 Daltons. Where the term percent (“%”) is used, the percent may be weight percent or molar percent.

In another aspect, the tdsRNA comprises a first ssRNA and a second ssRNA and each of these first ssRNA or second ssRNA may contain one or more strand breaks.

In another aspect, the tdsRNA has the property that greater than about 90%, greater than 95%, greater than 98%, greater than 99%, or 100% of the bases of the RNA are in a double-stranded configuration.

In any aspect, the tdsRNA may be in a therapeutic composition comprising, for example, a tdsRNA, and a pharmaceutically acceptable excipient (carrier).

One embodiment of tdsRNA is directed to rintatolimod, which is a tdsRNA of the formula rIₙ•r(C₁₂U), and which is also denoted by the trademark AMPLIGEN®.

In a preferred embodiment, the tdsRNA are of the general formula rIₙ•r(C₁₁₋₁₄, U)ₙ and are described in U.S. Pat. Nos. 4,024,222 and 4,130,641 (which are incorporated by reference herein) or synthesized according to this disclosure.

In the case where the tdsRNA is rAₙ•rUₙ, the tdsRNA may be matched (i.e., not in mismatched form).

tdsRNA (e.g., Rintatolimod) has undergone extensive clinical and preclinical testing. It has been well-tolerated in clinical trials enrolling over 1,200 patients with over 100,000 doses administered and there have been no drug-related deaths. Two placebo-controlled, randomized studies show no increase in serious adverse events compared to placebo. Favorable safety profiles have been seen for intraperitoneal, intravenous, and intranasal routes of administration of tdsRNA.

### 4.1 Length of tdsRNA

The length of the tdsRNA, may be represented by bases for one strand of the tdsRNA or in basepairs for both strands for the tdsRNA. It is understood that in some embodiments that not all of the bases (e.g., U and I) are in basepaired configuration. For example, rU bases do not pair as well as rC bases to inosine.

The length of the tdsRNA may be measured by (1) bases or basepairs, (2) molecular weight which is the weight of the double-stranded tdsRNA (e.g., Daltons) or (3) turns of the double-stranded RNA. These measurements can be easily interconverted. For example, it is generally accepted that there are about 629 Daltons per base pair.

“n” represents length in units of basepair or basepairs (abbreviated as bp regardless of whether it is singular or plural) for double-stranded nucleic acid. “n” can also represent bases for single-stranded RNA. Because “bp” represents singular or plural, it is the same as “bps” which is another representation of basepairs.

The tdsRNA can have the following values for its length “n” (in bases for single strand or basepairs for double strands): 4-5000, 10-50, 10-500, 10-40,000, 40-40,000, 40-50,000, 40-500, 50-500, 100-500, 380-450, 400-430, 400-800 or a combination thereof. Expressed in molecular weight, the tdsRNA may have the following values: 30 kDa to 300 kDa, 250 kDa to 320 kDa. 270 kDa to 300 kDa or a combination thereof. Expressed in helical turns, the tdsRNA may have 4.7 to 46.7 helical turns of duplexed RNA, 30 to 38 helical turns of duplexed RNA, 32 to 36 helical turns of duplexed RNA or a combination thereof.

The length may be an average basepair, average molecular weight, or an average helical turns of duplexed RNA and can take on integer or fractional values.

### 4.2 Rugged dsRNA (a Form of tdsRNA)

Rugged dsRNA is a tdsRNA that is resistant to denaturation under conditions that are able to separate hybridized poly(riboinosinic acid) and poly(ribocytosinic acid) strands (that is, rIₙ•rCₙ strands). See, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly(riboinosinic acid) and poly(ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃₅uracilic acid). Further, the single strand may be partially hybridized to an opposite strand comprised of poly(riboinosinic acid). In another aspect, rugged dsRNA may be an isolated double-stranded ribonucleic acid (dsRNA) which is resistant to denaturation under conditions that are able to separate hybridized poly(riboinosinic acid) and poly(ribocytosinic acid) strands.

In another aspect, Rugged dsRNA, has at least one of the following: r(Iₙ)•r(C₄₋₂₉U)ₙ, r(Iₙ)•r(C₁₂U)ₙ, r(Iₙ)•r(C₁₁₋₁₄U)ₙ, r(Iₙ)•r(C₃₀U)ₙ, or r(Iₙ)•r(C₃₀₋₃₅U)ₙ. Iₙ another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

### 4.3 Stabilizing Polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

### 4.4 Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

### 4.5 Carrier or Vehicle

Suitable agents may include a suitable carrier or vehicle for intranasal mucosal delivery. As used herein, the term “carrier” refers to a pharmaceutically acceptable solid or liquid filler, diluent or encapsulating material. In one aspect, the carrier is a suitable carrier or vehicle for intranasal mucosal delivery including delivery to the air passages and to the lungs of a subject.

A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories may be found in the U.S. Pharmacopeia National Formulary, 1857-1859, (1990). One commonly used pharmaceutically acceptable carrier is phosphate-buffered saline (PBS).

### 5. Administration Route

In a preferred embodiment, all of this disclosure (administrations, formulations, medicaments, compositions, dosages) relates to and describes at least to their application to a subject that is human. Additional non-human subjects are described below.

The pharmaceutical composition comprising one or more active agents (e.g., tdsRNA) of this disclosure may be administered to a subject by any local or systemic route or method known in the art. The preferred route may vary with the age, condition, gender, or health status of the subject; the nature of the disease, the number and severity of symptoms, chosen active ingredient, or the presence of other pathological conditions.

The most preferred methods include intravenous administration; intraperitoneal administration; or intranasal administration (including, e.g., breathing through the mouth or airway—e.g., through a stoma made by tracheostomy). Intravenous administration or intraperitoneal administration is commonly performed with a needle. Other administration methods include, at least, intradermal administration; subcutaneous administration; intramuscular administration; intraperitoneal administration; intracranial administration; intravesical administration; oral administration (e.g., and including through the mouth and/or by breathing through the mouth); topical administration; inhalation administration; aerosol administration; intra-airway administration; tracheal administration; bronchial administration; instillation administration; bronchoscopic instillation administration; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and a combination thereof. It is noted where more than one active ingredient (e.g., different tdsRNAs, etc.) is administered; the active ingredients may be administered by the same route or different routes.

Some forms of administration (administering) may be described by one or more of the above categories and some administration methods may be grouped differently or may be referred to by broader terms. For example, enteral administration may refer to oral administration, feeding tube administration, or enema administration; topical administration may be by a device such as a nebulizer for inhalation through the respiratory system, by skin patch acting epicutaneously or transdermally, or by suppository methods. Parenteral administration may take the form of subcutaneous administration, intravenous administration, intramuscular administration, intradermal administration, or intraperitoneal administration; buccal administration, sublingual administration, transmucosal administration; inhalation administration, instillation administration, instillation administration, intranasally administration, instillation administration, or intratracheal administration.

Nasal administration refers to any administration through the airway and comprises pulmonary airway administration. Nasal administration may include administration to the airway through the mouth (e.g., through breathing through the mouth or through a stoma made by tracheostomy).

Nasal administration includes administration to a tissue of the airway. This includes a tissue selected from the group consisting of: airway tissue; nose tissue; oral tissue; alveoli tissue; pharynx tissue; trachea tissue; bronchi tissue; carina tissue; bronchi tissue; bronchioles tissue; lung tissue; tissue in the lobe of a lung; alveoli tissue; nasal passage tissue; nasal epithelium tissue; larynx tissue; bronchi tissue; inhalation tissue; and a combination thereof. It follows that nasal administration may include administration to cells and tissues such as: an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; a sinus cell; and a combination thereof.

Administration may also be from any known delivery system. A delivery system may be selected from the group consisting of: a pill, a capsule, a needle, a cannula, an implantable drug depot, an infusion system (e.g., a device similar to an insulin pump); a nebulizer; a sprayer; a nasal pump; a squeeze bottle; a nasal spray; a syringe sprayer, a plunger sprayer (a syringe providing pressure to an attached sprayer or nozzle); a nasal aerosol device; a controlled particle dispersion device; a nasal aerosol device; a nasal nebulization device; a pressure-driven jet nebulizer; an ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

### 5.1 Formulations and Dosage

Formulations for administration (i.e., pharmaceutical compositions) may include a pharmaceutically acceptable carrier with the tdsRNA.

Pharmaceutical carriers include suitable non-toxic vehicles in which a composition of the disclosure is dissolved, dispersed, impregnated, or suspended, such as water or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants, softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

A liquid carrier may be present in the composition in a concentration effective to serve as a suitable vehicle for the compositions of the present disclosure. In general, the carrier is used in an amount of about 40 to about 98 wt. %, or about 50 to about 98 wt. % of the composition. Preferred forms of compositions are compositions for use as nasal sprays.

The liquid carrier may be water or any other suitable liquid, solvent, or mixture thereof. The water may contain suitable buffering agents to result in a pH wherein the particular antigen is delivered optimally, or it may contain other carriers, such as glycerin, propylene glycol, polyethylene glycols of various sizes, amino acid modifiers, such as arginine and the like, and other suitable soluble excipients, as is known to those who are proficient in the art of compounding or pharmaceutics. One preferred liquid carrier is phosphate-buffered saline (PBS).

The tdsRNA may be a combination or any subset of dsRNA described above (e.g., formula (1) to formula (5)). It is understood that in one aspect, tdsRNA may comprise a combination of all of the examples of tdsRNA described above or any subset of the above examples. With respect to the subsets, the specific exclusion of one or more specific embodiment of tdsRNA is also envisioned. As nonlimiting examples, tdsRNA may comprise any of the following: (A) all of the examples of tdsRNA as described above, (B) all of the examples of tdsRNA described above but without rIₙ•r(C₁₁₋₁₄U)ₙ, (C) Rugged dsRNA, (D) rIₙ•r(C₁₂U)ₙ, (E) tdsRNA as described above but without rIₙ•r(C₁₁₋₁₄U)ₙ and without Rugged dsRNA, (F) rIₙ•r(C₁₂U)ₙ, and Rugged dsRNA; or (G) rIₙ•r(C₁₁₋₁₄U)ₙ and Rugged dsRNA.

### 6. Additional Agents

### 6.1 Interferons

One optional component of the composition is interferon. As used herein, the term “interferon” (abbreviated “IFN”) refers collectively to type 1 and type 2 interferons and including deletion, insertion, or substitution variants thereof, biologically active fragments thereof, and allelic forms thereof. As used herein, interferon refers collectively to type 1 and type 2 interferons. Type 1 interferon includes interferons alpha, beta, omega and their subtypes. Human interferon alpha has at least 14 identified subtypes while interferon beta has 3 identified subtypes.

The interferon may be at least one selected from the group consisting of: interferon, interferon mixture, Alferon, alpha-interferon species, recombinant or natural interferon alpha, recombinant or natural interferon alpha 2a, recombinant or natural interferon beta, recombinant or natural interferon beta 1b, recombinant, and natural interferon gamma.

The interferon is optionally an alpha-interferon. One preferred alpha interferon is ALFERON N Injection® the only approved natural, multi-species, α-interferon available in the United States. Reverse phase HPLC studies show that ALFERON N Injection® is a consistent mixture of at least seven species of alpha interferon (α2, α4, α7, α8, α10, α16 and α17). This natural-source interferon has unique antiviral properties distinguishing it from genetically engineered interferons. The high purity of ALFERON N Injection® and its advantage as a natural mixture of seven interferon species, some of which, like species 8b, have greater antiviral activities than other species, for example, species 2b, which is the only component of INTRON A®. The superior antiviral activities, for example, in the treatment of chronic hepatitis C virus (HCV) and HIV infection, and tolerability of ALFERON N Injection® compared to other available recombinant interferons, such as INTRON A® and ROFERON A®, have been reported.

The interferon may be interferon species purified as a mixture of at least seven species of alpha-interferon produced by human white blood cells. The seven species may be, for example, interferon alpha 2; interferon alpha 4; interferon alpha 7; interferon alpha 8; interferon alpha 10; interferon alpha 16; and interferon alpha 17.

The recommended dosage of the components will depend on the clinical status of the patient and the experience of the clinician in treating similar conditions. As a general guideline, a dosage of ALFERON N Injection® utilized for systemic infections is 3 IU/pound to 10 million IU/pound (e.g., subcutaneous injection) three times weekly. Experience to date is with dosages above 3 IU/lb. of patient body weight. Oral α-interferon (ALFERON LDO®) has been administered as a liquid solution in the range of 500-10,000 IU/day and calculated on the basis of a 150-pound human this is from 3.3 to 66.0 IU/lb. per day. In one preferred embodiment, beneficial results are obtained at dosage levels of α-interferon in excess of 450 IU, that is greater than 3 IU/pound body weight. A healthcare provider would be able, however, to determine the optimal dose and schedule of low dose oral α-interferon (or any interferon) to achieve a desired antiviral effect.

### 6.2 Absorption-Promoting Agents

Suitable agents may include any suitable absorption-promoting agents. The suitable absorption-promoting agents may be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacyl methyl sulfoxide, azone (1-dodecylazacycloheptan-2-one or laurocapram), sodium lauryl sulfate, oleic acid, and the bile salts, may be employed to enhance mucosal penetration of the tdsRNA. In additional aspects, surfactants (e.g., polysorbates) are employed as adjunct compounds, processing agents, or formulation additives to enhance intranasal delivery of the tdsRNA.

### 6.3 Delivery-Enhancing Agents

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream) of tdsRNA or other biologically active compound(s).

In one aspect, enhancement of intranasal delivery can thus occur by any of a variety of mechanisms, for example by increasing the diffusion, transport, persistence or stability of tdsRNA, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

### 6.4 Mucolytic or Mucus Clearing Agents

In another embodiment, the present formulations may also comprise other suitable agents such as mucolytic and mucus-clearing agents. The term “mucolytic and mucus-clearing agents,” as used herein, refers to any agents which may serve to degrade, thin or clear mucus from intranasal mucosal surfaces to facilitate absorption of intranasally administered biotherapeutic agents including tdsRNA. Based on their mechanisms of action, mucolytic and mucus clearing agents can often be classified into the following groups: proteases (e.g., pronase, papain) that cleave the protein core of mucin glycoproteins, sulfhydryl compounds that split mucoprotein disulfide linkages, and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine. Other effective agents that reduce mucus viscosity or adhesion to enhance intranasal delivery according to the methods of the disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

### 6.5 Ciliostatic Agents

In another embodiment, the present formulations may comprise ciliostatic agents. As used herein, the term “ciliostatic agents” refers to any agents which are capable of moving a layer of mucus along the mucosa to removing inhaled particles and microorganisms. For use within these aspects of the disclosure, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance the delivery of tdsRNA and other biologically active agents without unacceptable adverse side effects.

Within more detailed aspects, a specific ciliostatic factor may be employed in a combined formulation or coordinate administration protocol with tdsRNA, and/or other biologically active agents disclosed herein. Various bacterial ciliostatic factors isolated and characterized in the literature may be employed within these embodiments of the disclosure. Ciliostatic factors from the bacterium Pseudomonas aeruginosa include a phenazine derivative, a pyo compound (2-alkyl-4-hydroxyquinolines), and a rhamnolipid (also known as a hemolysin).

### 6.6 Penetration or Permeation-Promoting Agent

In another embodiment, the intranasal mucosal therapeutic and prophylactic formulations of the present disclosure may be supplemented with any suitable penetration-promoting agent that facilitates absorption, diffusion, or penetration of tdsRNA across mucosal barriers. The penetration promoter may be any promoter that is pharmaceutically acceptable. Thus, another aspect relates to compositions comprising tdsRNA and one or more penetration-promoting agents selected from sodium salicylate and salicylic acid derivatives (acetyl salicylate, choline salicylate, salicylamide, etc.), amino acids and salts thereof (e.g., monoaminocarboxlic acids such as glycine, alanine, phenylalanine, proline, hydroxyproline, etc., hydroxyamino acids such as serine, acidic amino acids such as aspartic acid, glutamic acid, etc., and basic amino acids such as lysine, etc.—inclusive of their alkali metal or alkaline earth metal salts), and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

Also provided as penetration-promoting agents within the methods and compositions of the disclosure are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidones carboxylic acid esters, N-alkylpyrrolidones, proline acyl esters, and the like.

Non-limiting examples of other permeation enhancers useful in the instant disclosure are the simple long-chain esters that are Generally Recognized As Safe (GRAS) in the various pharmacopeial compendia. These may include simple aliphatic, unsaturated or saturated (but preferably fully saturated) esters, which contain up to medium-length chains. Non-limiting examples of such esters include isopropyl myristate, isopropyl palmitate, myristyl myristate, octyl palmitate, and the like. The enhancers are of a type that is suitable for use in a pharmaceutical composition. The artisan of ordinary skill will also appreciate that those materials that are incompatible with or irritating mucous membranes should be avoided.

For nasal administration, the enhancer is present in the composition in a concentration effective for enhancing penetration of the pharmaceutically active agent that is to be delivered through the nasal mucosa. Various considerations should be taken into account in determining the amount of enhancer to use. Such considerations include, for example, the amount of flux (rate of passage through the membrane) achieved and the stability and compatibility of the components in the formulations. The enhancer is generally used in an amount of about 0.001 to about 40 (w/w) % of the composition. Specific ranges include, about 0.01% to about 30 (w/w), about 0.1 to about 25% (w/w), about 1% to about 15% (w/w), about 5 to 10% (w/w). Alternatively, the amount of the enhancer may range from about 1.0 to about 3% (w/w) or about 10 to about 20% (w/w).

In forming an emulsion in which the water-insoluble enhancer is a normally solid material, the enhancer is dissolved in a suitable solvent. If the enhancer is a normally liquid material that is water-immiscible, a suitable solvent for the enhancer may or may not be used, as appropriate. In certain embodiments, the enhancer is dissolved, dispersed, suspended, or solubilized in a suitable solvent(s) such as alcohols, oils, glycerol, ethylene glycol, propylene glycol, hexane, acetone, freon, water, other polar or non-polar solvents, or a mixture, which is then added to a composition comprising an effective amount of the desired antigen admixed with a pharmaceutical carrier. In some cases, when the enhancers are in liquid form, a “neat” solution of enhancer can be directly incorporated in the antigen, pharmaceutical carrier, and enhancer mixture, in which the concentration of enhancer ranges from about 0.1% to about 50% (w/w).

Any of the above permeation enhancers are useful, especially in nasal administration.

### 6.7 Vasodilator or Vasoconstrictor Agents

In another embodiment, the present formulation may also comprise other suitable agents such as vasodilator agents. As used herein, the term “vasodilator agents” refers to any agents which are vasoactive. A vasodilator agent may function within the disclosure to modulate the structure and physiology of the submucosal vasculature, increasing the transport rate of tdsRNA, and other biologically active agents into or through the mucosal epithelium and/or to specific target tissues or compartments (e.g., the systemic circulation). Vasodilator agents for use within the disclosure typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, alpha-adrenergic and imidazole receptor antagonists, beta-1-adrenergic agonists, phosphodiesterase inhibitors, cicosanoids and NO donors. Within certain methods and compositions of the disclosure, a selected vasodilator agent may be coordinately administered (e.g., systemically or intranasally, simultaneously or in combinatorically effective temporal association) or combinatorically formulated with tdsRNA and other biologically active agent(s) in an amount effective to enhance the mucosal absorption of the active agent(s) to reach a target tissue or compartment in the subject.

In another embodiment, the present formulation may also comprise other suitable agents such as vasoconstrictor agents. As used herein, the term “vasoconstrictor agents” refers to any substances which may cause vasoconstriction. Vasoconstrictor agents may also be called vasoconstrictors, vasopressors, or simply “pressors.” Vasoconstrictor agents may usually cause an increase in systemic blood pressure, but when they are administered in specific tissues, localized blood flow may be reduced. The extent of vasoconstriction may be slight or severe depending on the substance of vasoconstrictor agents. Many vasoconstrictor agents may also cause pupil dilation. Vasoconstrictor agents may include any suitable substances such as antihistamines, decongestants and stimulants that are used to treat ADHD. Suitable vasoconstrictor agents have been previously described by Dhuria, Hanson, et al. (Dhuria, Hanson, et al., 2009).

### 6.8 Rnase Inhibitory Agent and Enzyme Inhibitor

In some embodiments, for example, nasal vaccines, the disclosure encompasses the delivery of a protein, peptide or another nucleic acid in addition to tdsRNA. Therefore, the compositions of the present disclosure may contain an enzyme inhibitor. As is well known to practitioners in nucleic acid, peptide and protein biochemistry, these biopolymers tend to be very sensitive to the presence of enzymes, such as RNase and proteolytic enzymes, that rapidly degrade the biopolymer when present in even minute amounts. Typical enzyme inhibitors that are commonly employed and that may be incorporated into the present disclosure may be, for example, leupeptin, aprotinin, and the like. Enzyme inhibitors also include nuclease inhibitors such as DNase inhibitors and RNase inhibitors. RNase inhibitors are commonly used as a precautionary measure in enzymatic manipulations of RNA to inhibit and control RNase. These are commercially available from a number of sources such as, for example, Invitrogen (SUPERase, In RNase Inhibitor, RNaseOUT, RNAsecure, and RNase Inhibitor).

### 6.9 Selective Transport-Enhancing Agents

In another embodiment, the present formulation may also comprise other suitable agents such as selective transport-enhancing agents. As used herein, the term “selective transport-enhancing agent” refers to any agent that facilitates transport of tdsRNA and/or one or more biologically active agents including vaccines. The compositions and delivery methods of the disclosure may optionally incorporate a selective transport-enhancing agent that facilitates transport of one or more biologically active agents. These transport-enhancing agents may be employed in a combinatorial formulation or coordinate administration protocol with tdsRNA disclosed herein, to coordinately enhance the delivery of one or more additional biologically active agent(s). Alternatively, the transport-enhancing agents may be employed in a combinatorial formulation or coordinate administration protocol to directly enhance mucosal delivery of tdsRNA, with or without enhanced delivery of an additional biologically active agent.

Exemplary selective transport-enhancing agents for use within this aspect of the disclosure may include, but are not limited to, glycosides, sugar-containing molecules, and binding agents such as lectin binding agents, and stabilizers. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the disclosure. Certain bioadhesive ligands for use within the disclosure will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more tdsRNA or functionally equivalent fragment proteins, analogs, mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the disclosure.

Additional intranasal mucosal delivery-enhancing agents that are useful within the coordinated administration and processing methods and combinatorial formulations of the disclosure may also include, but are not limited to, mixed micelles, enamines, nitric oxide donors (e.g., S-nitroso-N-acetyl-DL-penicillamine, NOR1, NOR4—which are preferably co-administered with a nitric oxide scavenger such as carboxy-PITO or diclofenac sodium), sodium salicylate, glycerol esters of acetoacetic acid (e.g., glyceryl-1,3-diacetoacetate or 1,2-isopropylideneglycerine-3-acetoacetate), and other release-diffusion or intra- or trans-epithelial penetration-promoting agents that are physiologically compatible for intranasal mucosal delivery. Other absorption-promoting agents may be selected from a variety of carriers, bases and excipients that enhance mucosal delivery, stability, activity or trans-epithelial penetration of the tdsRNA. These include, inter alia, cyclodextrins and beta-cyclodextrin derivatives (e.g., 2-hydroxypropyl-beta-cyclodextrin and heptakis(2,6-di-O-methyl-beta-cyclodextrin). These compounds, optionally conjugated with one or more of the active ingredients and further optionally formulated in an oleaginous base, enhance bioavailability in the intranasal mucosal formulations. Yet additional absorption-enhancing agents adapted for intranasal mucosal delivery may also include medium-chain fatty acids, including mono- and diglycerides (e.g., sodium caprate—extracts of coconut oil, CAPMUL), and triglycerides (e.g., amylodextrin, Estaram 299, Miglyol 810).

### 6.10 Stabilizing Delivery Vehicle, Carrier, Support or Complex-Forming Species

In another embodiment, the present formulation may also comprise other suitable agents such as a stabilizing delivery vehicle, carrier, support or complex-forming species. The coordinate administration methods and combinatorial formulations of the instant disclosure may optionally incorporate effective lipid or fatty acid-based carriers, processing agents, or delivery vehicles, to provide improved formulations for mucosal delivery of tdsRNA or functionally equivalent fragment proteins, analogs and mimetics, and other biologically active agents. For example, formulations and methods for mucosal delivery can comprise one or more of these active agents, such as a peptide or protein, admixed or encapsulated by, or coordinately administered with, a liposome, mixed micellar carrier, or emulsion, to enhance chemical and physical stability and increase the half-life of the biologically active agents (e.g., by reducing susceptibility to proteolysis, chemical modification and/or denaturation) upon mucosal delivery.

Within certain aspects of the disclosure, specialized delivery systems for biologically active agents may comprise small lipid vesicles known as liposomes or micelles. These are typically made from natural, biodegradable, non-toxic, and non-immunogenic lipid molecules, and can efficiently entrap or bind drug molecules, including peptides and proteins, into, or onto, their membranes. The attractiveness of liposomes as a nucleic acid delivery system is increased by the fact that the encapsulated tdsRNA can remain in their preferred aqueous environment within the vesicles, while the liposomal membrane protects them against nuclease and other destabilizing factors.

Additional delivery vehicles carrier, support or complex-forming species for use within the disclosure may include long and medium-chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the disclosure, long-chain fatty acids, especially fusogenic lipids (unsaturated fatty acids and monoglycerides such as oleic acid, linoleic acid, linoleic acid, monoolein, etc.) provide useful carriers to enhance mucosal delivery of tdsRNA, and other biologically active agents disclosed herein. Medium-chain fatty acids (C₆ to C₁₂) and monoglycerides have also been shown to have enhancing activity in intestinal drug absorption and can be adapted for use within the mucosal delivery formulations and methods of the disclosure. In addition, sodium salts of medium and long-chain fatty acids are effective delivery vehicles and absorption-enhancing agents for mucosal delivery of biologically active agents. Thus, fatty acids can be employed in soluble forms of sodium salts or by the addition of non-toxic surfactants, e.g., polyoxyethylated hydrogenated castor oil, sodium taurocholate, etc. Other fatty acid and mixed micellar preparations that are useful within the disclosure include, but are not limited to, Na caprylate (C8), Na caprate (C10), Na laurate (C12) or Na oleate (C18), optionally combined with bile salts, such as glycocholate and taurocholate.

### 7. Administration (Delivery)

Administration to the subject or administering to the subject may be in any known form including: systemic administration; intravenous administration; intradermal administration; subcutaneous administration; intramuscular administration; intranasal administration (e.g., including pulmonary airway administration); intranasal administration and oral administration; intraperitoneal administration; intracranial administration; intravesical administration; oral administration (e.g., and including through the mouth and/or by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration.

Intranasal administration may be administering to nasal passages; administering to nasal epithelium; administering to lung; administering by inhalation; administering to the larynx; administering to bronchi; administering to alveoli; administering by inhalation; administering by nasal instillation; and a combination thereof.

Administering or administration may be administering to at least one tissue or cell selected from the group consisting of: an airway tissue; nose tissue; oral tissue; alveoli tissue; pharynx tissue; trachea tissue; bronchi tissue; carina tissue; bronchi tissue; bronchioles tissue; lung tissue; lobe of a lung tissue; alveoli tissue; nasal passage tissue; nasal epithelium tissue; larynx tissue; bronchi tissue; inhalation tissue; an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; and a sinus cell.

As another example, administering may be performed by a delivery system or medical device comprising the tdsRNA. The delivery system or medical device may be a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhaler; a dry powder inhalation device; an instillation device; an intranasal instillation device; an intravesical instillation device; a swab; a pipette; a nasal irrigation device; a nasal rinse; an aerosol device; a metered aerosol device; a pressurized dosage device; a powdered aerosol device; a spray aerosol device; a spray device; a metered spray device; a suspension spray device; and a combination thereof. The liquid compositions are particularly suited for nasal administration.

### 8. Nasal Compositions

In one embodiment, a composition for enhancing intranasal delivery includes a combination of tdsRNA and active compounds prepared for nasal delivery. The combination of tdsRNA and active compounds may be applied in a subsequent manner or a simultaneous manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery.

### 8.1 Nasal Formulations

In one aspect, the present disclosure relates to formulations for nasal delivery of tdsRNA. In one aspect, tdsRNA is the sole active compound and may be free of any other active compounds. In another aspect, the tdsRNA may be co-administered with one or more additional active compounds.

Each of the agents and chemicals described herein, including any combinations thereof, may be added to a tdsRNA for any form of administration, including nasal administration, to a subject.

### 8.2 Compositions and Methods that are Generally Applicable and Particularly Applicable for Nasal Administration

In one embodiment, a composition for enhancing intranasal delivery includes tdsRNA and optionally active compounds prepared for nasal delivery. The combination of tdsRNA and active compounds may be applied in a subsequent (sequential) manner or a simultaneous (parallel) manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery. The composition may be in solid, liquid or any other form such as gels and liposomes.

A composition of the disclosure (e.g., tdsRNA) that is used in nasal administration is considered a nasal composition. Compositions of the disclosure are not limited to nasal administration. That is, any composition of the disclosure may be used as a nasal composition. Similarly, nasal compositions may be used for any other purposes such as non-nasal administration.

Simultaneous administration (also called parallel administration) may also comprise the administration of two or more compositions at the same time. For example, two or more separate nasal nozzles and sprayers can each dispense a different composition for simultaneous administration. Simultaneous administration may also dispense compositions of different forms. For example, a dry powder and a liquid may be dispensed together in separate sprayers at the same time.

Each of the agents and chemicals described herein, including any combinations thereof, may be administered together with a composition of the disclosure (e.g., tdsRNA), nasally or otherwise, to a subject. Non-limiting examples of other compounds for nasal administration include RNA, DNA, adjuvants, proteins, interferons, or parts thereof.

We note that tdsRNA is stable as a solid or dissolved in water and therefore any additional component, such as phosphate-buffered saline, is optional. Other components may benefit from additional ingredients described herein.

In certain embodiments, the therapeutic agent is administered with an agent that disrupts, e.g., transiently disrupts, tight junctions, such as EGTA (see U.S. Pat. No. 6,855,549).

### 9. Specific Examples of Compositions

Aerosol compositions can be made with liquid and dried compositions of the disclosure to be administered via inhalation. These aerosol compositions can be placed into pressurized acceptable propellants, such as dichlorodifluoromethane, propane, and nitrogen. Compositions may be formulated as pharmaceuticals for non-pressured preparations, such as in a nebulizer or an atomizer. For compositions to be administered from multiple-dose containers, antimicrobial agents can be added.

Liquid solutions may be suitable for any administration including nasal administration. Liquid compositions may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The composition of the disclosure can be administered in a physiologically acceptable diluent in a pharmaceutically acceptable carrier, such as a sterile liquid or mixture of liquids, including water, saline, phosphate buffered saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The compositions may be formulated as dry, semidry, or liquid particles. The particulate pharmaceutical composition may optionally be combined with a carrier to aid in dispersion or transport. A suitable carrier such as a sugar (i.e., dextrose, lactose, sucrose, trehalose, mannitol) may be blended with the active compound or compounds in any suitable ratio.

Specific examples of composition forms include at least the following: aerosol of liquid, aerosol suspension of respirable solid, dry powder inhalants, metered-dose inhalants, liquid/liquid suspensions, emulsions, suspensions, oil in water emulsion, and water in oil emulsions.

In reference to particles or droplets, it is envisioned that a particle or a droplet may be a solid, a liquid, or other types of particle such as a gel, a liposome, and the like. Also, it is envisioned that a composition may be dispensed as one type of particle but is delivered to a subject as a second type of particle. For example, a composition may be dispensed as a liquid particle with a high evaporation rate such that the liquid is transformed into a solid by the time the particle reaches the subject.

Certain devices require the use of various compositions suitable for the dispensing of some compositions of the present disclosure. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified systems may also be prepared in different compositions depending on the type of chemical modification or the type of device employed.

Compositions suitable for use with a nebulizer may also include a buffer and a simple sugar (e.g., for stabilization of the composition and regulation of osmotic pressure). The carrier is typically water (and most preferably sterile, pyrogen-free water) or a dilute aqueous alcoholic solution, preferably made isotonic, but may be hypertonic with body fluids by the addition of, for example, sodium chloride. The nebulizer composition may also contain a surfactant to reduce or prevent surface-induced aggregation caused by atomization of the solution in forming the aerosol. Optional additives include preservatives if the composition is not made sterile, for example, methyl hydroxybenzoate, antioxidants, flavoring agents, volatile oils, buffering agents and surfactants.

Compositions for use with a metered-dose inhaler device may generally comprise a finely divided powder (a composition of the disclosure) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Compositions for dispensing from a powder inhaler device may comprise a finely divided dry powder containing a composition as described herein, and may also include a bulking agent, such as lactose, sorbitol, sucrose, or mannitol in amounts that facilitate dispersal of the powder from the device, e.g., 50 to 90% by weight of the composition. The composition may be prepared in particulate form with an average particle size of less than 10 mm (or microns), most preferably 0.5 to 5 mm, for the most effective delivery to the distal lung.

Non-limiting specific examples of nasal (pulmonary) administration include at least one or more of the administration methods such as oral administration (e.g., and including through the mouth and/or by breathing through the mouth); intranasal administration (e.g., by nose drops); inhalation administration; aerosol administration; intra-airway (e.g., tracheal or bronchial) administration; bronchoscopic instillation; intratracheal administration; mucosal administration; dry powder administration; respiratory administration; instillation administration.

Another example of nasal administration includes any deposition to any part of the airway, including, for example, by spray, by a swab, intratracheal deposition, intrabronchial deposition and bronchoscopic deposition, nasal rinse, nasal lavage, a temporary or permanent depot implant.

Administration by “inhalation” may be performed using a composition of the disclosure of a size sufficiently small to pass through the mouth or nose and larynx, past the oropharyngeal region, upon inhalation and into the bronchi and alveoli of the lungs. In general, particles (droplets, liquid or solid) ranging from about 1 to 10 microns in size (more particularly, less than about 5 microns in size) are respirable and suitable for administration by inhalation. The particles can be solid or liquid. In some embodiments, such preparations have a mean particle size of 4, 5, 6, 7, 8, 9, 10, 11, 12, or 13 microns.

In some embodiments, preparations for inhaled or aerosol delivery are formulated as a dry powder. In some embodiments, preparations for inhaled or aerosol delivery are formulated as a wet powder, for example through inclusion of a wetting agent. in some embodiments, the wetting agent is selected from the group consisting of water, saline, or other liquid of physiological pH. In some embodiment, the particles may be a liquid.

Administration by intranasal administration may be performed by particles of a larger size formulated and delivered to topically treat the nasal epithelium. Particles or droplets used for intranasal administration generally have a diameter that is larger than those used for administration by inhalation. For intranasal administration, a particle size in the range of 10-500 microns is preferred to ensure retention in the nasal cavity.

In some embodiments, particles for inhalation and particles for intranasal administration may be administered together. That is, particles of 1 to 500 microns are used. In some embodiments, particles of 1-10 or 1-13 microns are selected for or enriched. In other embodiments, particles of 10-500 microns, or 15 to 500 microns are selected for or enriched.

The compositions of the disclosure may be administered as a plurality of drops to the nasal or buccal cavity. A dose may be, for example, 1-100, 1-50, 1-20, 1-10, 1-5, drops.

In some embodiments, administering may comprise using a device that delivers a metered dosage of composition.

Aerosols of liquid particles of the compositions of the disclosure may be produced by any suitable means, such as with a nebulizer, pressure-driven jet nebulizer, an ultrasonic nebulizer, or other means. Aerosols of solid particles comprising the composition of the disclosure may likewise be produced with any solid particulate therapeutic aerosol generator. One illustrative type of solid particulate aerosol generator is an insufflator. Suitable compositions for administration by insufflation include finely comminuted powders which may be delivered by means of an insufflator or taken into the nasal cavity in the manner of a snuff. In the insufflator, the powder (e.g., a metered-dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the composition of the disclosure or of a powder blend comprising the composition and a suitable powder diluent, such as lactose, and an optional surfactant.

Another type of illustrative aerosol generator comprises a metered-dose inhaler. Metered-dose inhalers are pressurized aerosol dispensers, typically containing a suspension or solution composition of the tdsRNA in a liquefied propellant. During use these devices discharge the composition through a valve adapted to deliver a metered volume, typically from 10 μl to 200 μl, to produce a fine particle spray containing the tdsRNA. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidant and suitable flavoring agents.

The preferred route and mode of administration will vary with the condition and age of the recipient, the nature of the infection or condition, and the chosen active ingredient.

### 10. Nasal Administration Devices

A device or delivery system, encompassing a composition of the disclosure is also an embodiment.

The composition of the disclosure may be delivered by any nasal administration device or combination of devices. A combination refers to a composition that is both administered by two different devices or a device having the feature of two devices. Non-limiting examples of suitable devices that can be use individually or together include at least one selected from the group consisting of: a nebulizer; a sprayer (e.g., a spray bottle such as “Nasal Spray Pump w/Safety Clip, Pfeiffer SAP #60548; a squeeze bottle (e.g., bottle commonly used for nasal sprays, including ASTELIN (azelastine hydrochloride, Medpointe Healthcare Inc.) and PATANASE (olopatadine hydrochloride, Alcon, Inc.); a nasal pump spray (e.g., APTAR PHARMA nasal spray pump); a controlled particle dispersion devices (e.g., VIANASE electronic atomizer); a nasal aerosol device (e.g., ZETONNA nasal aerosol); a nasal nebulization device (e.g., EASYNOSE nebulizer, a pressure-driven jet nebulizer, or an ultrasonic nebulizer); a powder nasal delivery devices (e.g., OPTINOSE breath-powered nasal delivery device); an atomized nasal medication device (e.g., LMA MAD NASAL device); an instillation device; an inhalation device (e.g., an inhaler); a powder dispenser; a dry powder generator; an aerolizer (e.g., intrapulmonary aerosolizer or a sub-miniature aerosolizer, metered aerosol, powdered aerosol, spray aerosol); a spray; a metered spray; a metered dose inhalers (e.g., a propellant based metered-dose inhaler); a dry powder inhalation device; an intranasal instillation device; an intravesical instillation device; an insufflation device.

An application device for application to mucous membranes, such as, that of the nose, throat, and/or bronchial tubes (i.e., inhalation). This can be a swab, a pipette or a device for nasal irrigation, nasal rinse, or nasal lavage.

Another example is a syringe or plunger-activated sprayer. This could be, for example, a sprayer head (or nozzle) attached, for example, via a Luer lock, to a syringe. The syringe applies pressure to a composition that flows through the sprayer head and produces a spray or an aerosol.

### 10.1 More Specific Examples of Nasal Devices

Aerosol: A product that is packaged under pressure and contains therapeutically active ingredients that are released upon activation of an appropriate valve system. For use as aerosols, the compounds of the present disclosure in solution or suspension may be packaged in a pressurized aerosol container together with suitable propellants, for example, hydrocarbon propellants like propane, butane, or isobutane with conventional adjuvants. The materials of the present disclosure also may be administered in aerosol but in a non-pressurized form such as in a nebulizer or atomizer.

Metered Aerosol: A pressurized dosage form comprised of metered-dose valves, which allow for the delivery of a uniform quantity of spray upon each activation.

Powdered Aerosol: A product that is packaged under pressure and contains therapeutically active ingredients in the form of a powder, which are released upon activation of an appropriate valve system.

Spray aerosol: An aerosol product that utilizes a compressed gas as the propellant to provide the force necessary to expel the product as a wet spray.

Spray: A liquid minutely divided as by a jet of air or steam. Nasal spray drug products contain therapeutically active ingredients dissolved or suspended in solutions or mixtures of excipients in non-pressurized dispensers.

Metered spray: A non-pressurized dosage form consisting of valves that allow the dispensing of a specified quantity of spray upon each activation.

Suspension spray: A liquid preparation containing solid particles dispersed in a liquid vehicle and in the form of course droplets or as finely divided solids.

Some non-limiting specific examples of commercially available devices are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

One illustrative type of solid particulate aerosol generator is an insufflator. Suitable compositions for administration by insufflation include finely comminuted powders which may be delivered by means of an insufflator or taken into the nasal cavity in the manner of a snuff. In the insulator, the powder (e.g., a metered-dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the composition.

A second type of illustrative aerosol generator comprises a metered-dose inhaler. Metered-dose inhalers are pressurized aerosol dispensers, typically containing a suspension or solution composition of the active ingredient in a liquefied propellant. During use these devices discharge the composition through a valve adapted to deliver a metered volume, typically from 10 to 200 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidant and suitable flavoring agents.

It is noted that while some of the devices may dispense a liquid, the liquid may be a rapidly evaporating liquid that would turn into a dry powder before contact with a patient. Therefore, in effect, the spray can be considered a dry powder administration.

Any of the listed devices may be incorporated into an administration device embodiment of this disclosure.

### 11. Medicament and Dosage

In another aspect, a medicament (e.g., a pharmaceutical composition) containing the tdsRNA is provided. Optional other components of the medicament include excipients and a vehicle (e.g., aqueous buffer or water for injection) packaged aseptically in one or more separate containers (e.g., nasal applicator or injection vial). Further aspects will be apparent from the disclosure and claims herein.

### 11.1 Dosage for the Average Subject

The dosages are generally applicable to a subject as described in another section of this disclosure. In a preferred embodiment, the subject is human.

For a subject the dose of tdsRNA per day may be at least one selected from the group consisting of: 0.1 μg to 1,000,000 μg, 0.1 μg to 25,000 μg, 0.4 to 400,000 μg, 0.5 μg to 5,000 μg, 0.5 mg to 60 mg, 5 mg to 40 mg, 5 mg to 400 mg, 10 mg to 20 mg, 10 mg to 800 mg, 25 mg to 700 mg, 20 mg to 200 mg, 50 mg to 150 mg, 80 mg to 140 mg, and a combination thereof.

A subject may be a human of about 150 lb. or 70 Kg in weight, and the appropriate dosage per body weight may be calculated.

### 11.2 Dose in Kilogram Per Day

In another aspect, the tdsRNA is administered in a dose per day selected from the group consisting of 0.1 mg/kg, 0.2 mg/kg, 0.3 mg/kg, 0.5 mg/kg, 0.7 mg/kg, 0.8 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 0.1-1 mg/kg, 0.1-2 mg/kg, 0.1-3 mg/kg, 0.1-4 mg/kg, 0.1-5 mg/kg, 0.1-6 mg/kg, 0.1-7 mg/kg, 0.1-8 mg/kg, 0.1-10 mg/kg, 0.1-20 mg/kg, 0.2-3 mg/kg, 0.3-3 mg/kg, 0.4-3 mg/kg, 0.6-3 mg/kg, and 0.8-3 mg/kg.

### 11.3 Amount Per Unit Dose

The amount per unit dose of tdsRNA may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg, 5 mg/kg, 6 mg/kg, 8 mg/kg, 10 mg/kg.

### 11.4 Specific Examples

In one embodiment, the tdsRNA is administered at a dose from about 1 mg/kg to 10 mg/kg biweekly. As another example, the administration may be in 50-1400 milligrams every other day, leading to an average daily dosage of 25-700 milligrams per day. In one embodiment, the tdsRNA is administered at a dose from about 0.50 mg/kg to 10 mg/kg every other week. 50-1400 milligrams every other day, leading to an average daily dosage of 25-700 milligrams per day.

### 11.5 Dose Frequency

In certain embodiments, the tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, one dose every two weeks, one dose every three weeks, one dose every four weeks, and one dose every month.

### 11.6 Number of Doses and Dosing Period

In certain embodiments, the tdsRNA is administered as a single dose, in two doses, in three doses, in four doses, in five doses, or in 6 or more doses. In other embodiments, the dosage is continued indefinitely. Continuous dosage may be used under some circumstances, for example, if the subject is already using an insulin pump the tdsRNA may be admixed with the insulin.

A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, 1 month, 2 months, 3 months, 4 months, 6 months, 9 months or one year. In certain embodiments, multiple (for example, 2, 3, 4, 5, 6, 7, 8, 9, 10 or more) doses of a tdsRNA are administered to a subject in need of treatment. It is envisioned that for some subjects, the dosing periods may be 1 year, 2 years, 3 years, 5 years, 10 years or continuous.

### 11.7 Nasal Dosage

tdsRNA may be administered at the same dose in nasal administration as for any other form of administration. Nonlimiting specific examples of nasal administration (which is also applicable for any other form of administration) include a dose of 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof.

Unless otherwise specified, “composition,” “a composition,” or “the composition” includes, at least, a composition of the disclosure or includes at least tdsRNA. Compositions may be optionally filtered and sterilized to enhance safety, stability and solubility. The composition may be formulated to enhance the delivery method. For example, the formulation may be formulated to enhance i.v. delivery, intraperitoneal delivery or nasal delivery.

### 12. Subject or Patient

As used herein, a “subject” has the same meaning as a “patient” and is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include any animal such as civet cats, swine, cattle, horses, camels, cats, dogs, rodents, birds, bats, rabbits, ferrets, mink, snake, and the like. As used herein, the terms “patient” or “subject” are used interchangeably.

### 13. Devices and Kits

In another aspect, the present disclosure relates to and comprises a therapeutic device for intranasal delivery. In one embodiment, the therapeutic device may comprise any suitable devices charged with a preparation of tdsRNA and optionally, another biologically active agent such as a vaccine or antigen. These devices are described in more detail below.

### 14. Effective Amount: Therapeutically or Prophylactically Effective Amount

The compositions are delivered in effective amounts. The term “effective amount” refers to the amount necessary or sufficient to realize a desired biologic effect which is, for example, inhibiting, attenuating, preventing or at least reducing a symptom, a sign, a pathology of a neuroinflammatory disorder, or a risk thereof. In addition to the sample dosages and administration methods mentions, one of ordinary skill in the art can empirically determine the effective amount of the tdsRNA without necessitating undue experimentation. It is generally preferred that a maximum dose be used, that is, the highest safe dose according to medical judgment.

Effective dosage forms, modes of administration, and dosage amounts may be determined empirically, and making such determinations is within the skill of the art. It is understood by those skilled in the art that the dosage amount will vary with the route and mode of administration, the rate of excretion, the duration of the treatment, the identity of any other drugs (e.g., antiviral agent) being co-administered, the age, size, species of mammal (e.g., human patient), and other factors well known in the arts of medicine and veterinary medicine. In general, a suitable dose of any active agent disclosed herein or a composition containing the same will be that amount of the active agent or composition, which is the lowest dose effective to produce the desired effect. The desired effect may be to reduce the severity or duration of a symptom, a sign, or a pathology of a neuroinflammatory disorder such as, for example, Alzheimer's disease (AD); Parkinson's disease; Multiple Sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); and multi-system atrophy.

### 15. Other Aspects

Unless otherwise indicated, the term “Alzheimer's” or “Alzheimer” has the same meaning as “Alzheimer's disease;” the term “Parkinson's” or “Parkinson” has the same meaning as “Parkinson's disease;” the terms “disorder” and “disease” are used interchangeably.

In this specification, stating a numerical range, it should be understood that all values within the range are also described (e.g., one to ten also includes every value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim, the disclosure is also described by such claims reciting the transitional phrases “consisting essentially of” (i.e., allowing the inclusion of other elements to be within the scope of the claim if they do not materially affect the operation of the disclosure) or “consisting of” (i.e., allowing only the elements listed in the claim other than impurities or inconsequential activities which are ordinarily associated with the disclosure) instead of the “comprising” term. Any of these three transitions can be used to claim the disclosure.

An element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics.

While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### 16. Incorporation by Reference

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369, 7,439,349, 8,722,874 and 9,315,538. Iₙ case of conflict, the present application, including any definitions herein, will control.

### EXAMPLES

### Example 1 Selection of Study Population

This is a prospective study to determine the effects of tdsRNA on neuroinflammation symptoms (Alzheimer's disease symptoms) such as cognitive decline. Alzheimer's disease symptoms were studied because they are models for a disease/disorder related to neuroinflammation.

### Study Subjects

There were 92 subjects in the study, 45 subjects randomly assigned to Poly I: Poly C₁₂U and 47 subjects randomly assigned to placebo. Subjects were experiencing cognitive decline which may be a result of neuroinflammation. The 45 subjects in the Poly I: Poly C₁₂U group received 2038 doses of the study drug (Poly I: Poly C₁₂U which is rintatolimod) of 2160 scheduled doses, and the 47 subjects in the placebo group received 2141 of 2256 scheduled doses; the overall compliance rate for both treatment groups was 88%.

Each participant in the active treatment group received an average total dose of 17093 mg/subject of the 18400 mg of Poly I: Poly C₁₂U scheduled.

Exposure to Poly I: Poly C₁₂U is presented in the Table below.

Subject Exposure to Poly I: Poly C₁₂U (Safety Population)

| | Poly I:Poly C₁₂U 400 mg | Placebo |
|---|---|---|
| Exposure Category | (N = 45) | (N = 47) |
| Total number of treatments | 2160 | 2256 |
| scheduled | | |
| Total number of treatments | 2038 | 2141 |
| given | | |
| Total number of missed | 24 (1.2) | 28 (1.3) |
| doses, n (%) | | |
| Scheduled Total Poly I:Poly | 18400 | 0 |
| C₁₂U, Dosage, mg/subject | | |
| Average Total Poly I:Poly | 17093 | 0 |
| C₁₂U Dosage, mg/subject | | |
| Average treatment duration, | 22.6 | 22.8 |
| weeks | | |
| Subjects who completed the | 41 | 43 |
| study n | | |

### Example 2: Treatments Administered

Subjects were randomly assigned to receive Poly I: poly C₁₂U or placebo (matched to active treatment) administered as a 35-minute (±5 minutes) by i.v. infusion.

Identity of tdsRNA

The tdsRNA used for treatment is Polyriboinosinic: polyribocytidylic (12:1) uridylic acid (Poly I: Poly C₁₂U) is a synthetic, double-stranded ribonucleic acid, which contains regularly occurring regions of unmatched (non-hydrogen bonded) base pairs. It is a specific form of dsRNA in which U substitution in the cytidylic chain creates a region of non-hydrogen bonding in the molecular configuration.

Poly I: Poly C₁₂U is a biological response modifier, a relatively non-toxic derivative of poly(I)ₙ:poly(C)ₙ. Poly I:Poly C₁₂U has relatively short half-life of approximately 30 to 40 minutes compared with the >4 hour half-life of the parent poly(I)ₙ:poly(C)ₙ, as confirmed by previous clinical studies.

Poly I: Poly C₁₂U was reconstituted with sterile preservative-free water, resulting in a physiological salt solution with drug suitable for i.v., administration.

### Selection and Timing of Dose for Each Subject

This was a randomized study. Subjects were randomly assigned to Poly I: Poly C₁₂U 400 mg or placebo (matched to active treatment) administered as an i.v., infusion over 35 minutes (±5 minutes) to all subjects. Poly I: Poly C₁₂U 200 mg was administered twice weekly for 2 weeks (total of 4 doses), then Poly I: Poly C₁₂U 400 mg was administered twice weekly through to the end of the 24-week study period.

Karnofsky Performance Status (KPS) scores were measured weekly and SCL-90-R CD subscale scores (See, O'Donnell W E, DeSoto C B, Reynolds D M. A cognitive deficit subscale of the SCL-90-R. J Clin Psychol 1984 January; 40(1):241-46.) were measured on weeks 0 (right before treatment), 8, 16 and 24. Administration of tdsRNA was started on week zero after the baseline scores were measured.

The Eight Item Cognitive Deficit (CD) Subscale of SCL-90-R used are as follows (See, O'Donnell W E, DeSoto C B, Reynolds D M. A cognitive deficit subscale of the SCL-90-R. J Clin Psychol 1984 January; 40(1):241-46.).

| Item Number | Verbal Content | Symptom Dimension |
|---|---|---|
| 1 | Headaches | Somatization |
| 9 | Trouble Remembering | Obsessive/compulsive |
| 24 | Temper Outburst | Hostility |
| 38 | Do things slow | Obsessive/compulsive |
| 45 | Double Check | Obsessive/compulsive |
| 51 | Mind goes blank | Obsessive/compulsive |
| 55 | Trouble Remembering | Obsessive/compulsive |
| 87 | Wrong With Body | Psychoticism |

### Example 3: The Study

### Periods of the Study and Variables Assessed

This study was divided into 3 periods: baseline, treatment, and completion (termination). The frequency and timing of protocol-defined evaluations and procedures performed at specific time points during these periods included efficacy and safety. These are summarized below.

### Baseline Period

Eight Item Cognitive Deficit (CD) Subscale of SCL-90-R scores were evaluated and recorded by the investigator and stratification was performed prior to the initiation of study treatment.

### Treatment Period

During the Treatment Period, all subjects received an infusion of Poly I: Poly C₁₂U 200 mg or placebo (matched to active treatment) twice weekly for 2 weeks (for a total of 4 doses), then Poly I: Poly C₁₂U 400 mg or placebo was administered twice weekly until the end of the 24-week study period.

Protocol-specified evaluations and procedures were performed at specific time points. Those performed every 4 weeks included physical examination and vital signs including weight and pregnancy test for women of childbearing potential.

Clinical chemistry tests were performed every 8 weeks during the Treatment Period and included creatinine, electrolytes, a biochemical profile (calcium, phosphate, glucose, BUN, uric acid, cholesterol, total protein, albumin, bilirubin [total and direct], alkaline phosphatase, LDH, SGOT, and SGPT). Hematology tests were performed every 8 weeks during the Treatment Period and included a CBC including Hct, Hgb, WBC count with differential (neutrophils, lymphocytes, monocytes, eosinophils and basophils) and platelet count. Coagulation studies (PT and PTT) and urinalysis were performed every 8 weeks.

Concomitant medication and adverse events were collected and recorded weekly.

Every 8 weeks, SCL-90-R CD results were recorded.

### Completion/Termination Period

The Completion (Termination) Period occurred at Week 24 (or at early termination if a subject withdrew or was terminated from the study prematurely). Evaluations and procedures included physical examination, vital signs.

Clinical chemistry tests performed at the Completion (Termination) Period included creatinine, electrolytes, a biochemical profile (calcium, phosphate, glucose, BUN, uric acid, cholesterol, total protein, albumin, bilirubin [total and direct], alkaline phosphatase, LDH, SGOT, and SGPT). Hematology included a CBC including Hct, Hgb, WBC count with differential (neutrophils, lymphocytes, monocytes, eosinophils, and basophils) and platelet count. Coagulation studies (PT and PTT) and urinalysis were performed.

SCL-90-R CD were recorded.

### Neurocognitive Functional Status Using the Symptoms Checklist-90-Revised (SCL-90-R) Cognitive Deficit (CD) Subscale

Neurocognitive functional status was determined by the SCL-90-R CD Subscale. The SCL-90-R CD was performed at baseline, every eight weeks during the study, and at the Completion/Termination visit.

The cognition deficit (CD) within the SCL-90-R is a validated self-assessment instrument consisting of eight questions that require the subject to indicate the level of distress caused by concurrent neurocognitive disturbances. The eight specific questions that comprise the CD subscale of the SCL-90-R are embedded within a series of 90 other items. Assessments are based upon responses to questions posed indirectly. As the subject's cognitive ability is not based upon performance under contrived “testing” conditions, even with practice, the subject cannot falsely elevate estimates of cognitive ability.

Subjects reported the level of impairment produced by each of the 8 items on an ascending unitary scale of 0, 1, 2, 3, and 4; “4” reflected extreme impairment and “0” indicated the absence of impairment. The cognitive deficit score is the average of the subject's response to all 8 items. Accordingly, decreases in SCL-90-R CD scores from baseline to study completion indicate decreased cognitive impairment (i.e., clinical improvement).

### Sample Size and Power

It was determined that with 60 subjects per treatment group, a power analysis of ≥90% and a 2-sided Type I error of 5% (two-sided) would enable detection of a difference of ≥20% between treatment groups. (e.g., an increase from 50 to 60 the primary endpoint, the KPS score). p-Values were provided as appropriate. Statistical significance was declared if the 2-sided p-value was <0.05.

### Demographic and Baseline Characteristics

Overall, the majority of subjects were female (75%) and white (95.7%). The mean age of subjects was 41.7 years in the active treatment group and 38.9 years in the placebo group. There was a statistically significant (p=0.022) difference between the active treatment group and the placebo group in gender.

Demographic and baseline characteristics are presented in the table below.

Demographic and Baseline Characteristics (Safety Population)

| | Poly I:Poly | | | |
|---|---|---|---|---|
| | C₁₂U 400 mg | Placebo | Total | p- |
| Characteristic | (N = 45) | (N = 47) | (N = 92) | Value |
| Sex, n (%) | | | | 0.022ª |
| Male | 16 (35.6) | 7 (14.9) | 23 (25.0) | |
| Female | 29 (64.4) | 40 (85.1) | 69 (75.0) | |
| Age, years | | | | |
| n | 45 | 47 | 92 | |
| Mean | 41.7 | 38.9 | | 0.107^{b} |
| Min, Max | 25, 69 | 26, 54 | 25, 69 | |
| Race, n (%) | | | | 0.328ª |
| White | 44 (97.8) | 44 (93.6) | 88 (95.7) | |
| Non-white | 1 (2.2) | 3 (6.4) | 4 (4.4) | |
| (Black, | | | | |
| Oriental, | | | | |
| Hispanic) | | | | |

^{a}Chi-square test.
^{b}Student's t-test.

### Results

There was a statistically significant (p=0.036) improvement in mean change in SCL-90-R CD scores from baseline to Week 24 in the Poly I: Poly C₁₂U group compared with the placebo group. Decreases in mean SCL-90-R CD scores in the ITT Poly I: Poly C₁₂U group from baseline to Week 24 indicated a steady trend towards a decrease in cognitive impairment following treatment with study medication. The mean change (improvement) in score of −0.72 for the ITT Poly I: Poly C₁₂U group (28.3%) compared with the mean change (worsening) in score of 0.40 for the placebo group (15.9%) indicated the Poly I: Poly C₁₂U group improved more than 44% compared with the placebo group.

We observed improvements from baseline to Week 24 in SCL-90-R CD score, statistically significantly greater in the PolyI: Poly C₁₂U group than in the placebo group. There was a statistically significant (p=0.036) improvement in mean change in SCL-90-R CD scores from baseline to Week 24 in the Poly I: Poly C₁₂U group compared with the placebo group. Decreases in mean SCL-90-R CD scores in the ITT Poly I: Poly C₁₂U group from baseline to study completion indicated a steady trend towards a decrease in cognitive impairment following treatment with study medication. The mean change (improvement) in score of −0.72 for the ITT Poly I: Poly C₁₂U group (28.3%) compared with the mean change (worsening) in score of 0.40 for the placebo group (15.9%) indicated the Poly I: Poly C₁₂U group improved more than 44% compared with the placebo group.

A summary of the mean change in SCL-90-R CD scores from Baseline to Week 24 for the ITT Population is presented in the Table below.

Summary of Means and Mean Changes in Symptoms Checklist90 Revised Cognitive Deficit (SCL-90-R CD) Subscale Scores from Baseline to Week 24 of Treatment (ITT Population)

| | Poly I:Poly | | | | | | |
|---|---|---|---|---|---|---|---|
| | C₁₂U 400 mg | | | Placebo | | | |
| | (N = 45) | | | (N = 47) | | | |
| | | | Mean | | | Mean | p- |
| Time | Mean | | Change^{a} | Mean | | Change^{a} | Value |
| Baseline | | | | | | | |
| n | | 43 | | | 44 | | |
| Mean | 2.54 | | | 2.52 | | | 0.863^{b} |
| Week 24 | | | | | | | |
| n | | 43 | | | 44 | | |
| Mean | | | −0.72^{d} | | | 0.40^{e} | 0.036^{c} |

^{a}Mean Change: adjusted by least square means
^{b}Student's t-test.
^{c}An ANCOVA with baseline as covariate.
^{d}A decrease in the CD score indicates improvement in cognitive ability.
^{e}An increase in the CD score indicates a worsening in cognitive ability.
Abbreviations:
ANCOVA = analysis of covariance,
SCL-90-R CD = Symptoms Checklist 90 Revised Cognitive Deficit.

1. A method for treating, reducing, or preventing neuroinflammation in a subject in need thereof, comprising
administering to the subject a composition comprising a therapeutically
effective amount of therapeutic double stranded RNA (tdsRNA);
wherein the tdsRNA is at least one selected from the group consisting of: 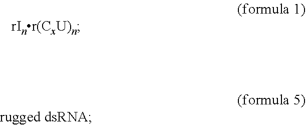
wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

2. The method of claim 1, wherein the neuroinflammation is at least one selected from the group consisting of:
a neuroinflammation disorder; a neuroinflammation disorder symptom; a neuroinflammation disorder pathology; and a neuroinflammation disorder sign.

3. The method of claim 1,
wherein the neuroinflammation is at least one selected from the group consisting of: neuroinflammation; chronic neuroinflammation; depression; schizophrenia; Alzheimer's disease (AD); Parkinson's disease; multiple sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); multi-system atrophy; acute disseminated encephalomyelitis (ADEM); acute optic neuritis (AON); transverse myelitis; and Neuromyelitis Optica (NMO).

4. The method of claim 1, further comprising a step before the administering step of determining that the subject has or is at risk of developing at least one selected from the group consisting of:
neuroinflammation disorder; neuroinflammation disorder symptom;
neuroinflammation disorder pathology; and neuroinflammation disorder sign.

5. The method of claim 1, wherein the disorder symptom, disorder pathology, or disorder sign is at least one selected from the group consisting of:
amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; a high or increasing score on a Cognitive Deficit (CD) subscale of the SCL-90-R Scale; microglial activation; astrocyte activation; elevated IL-6 (interleukin-6); elevated IL-23 (interleukin-23); elevated IL-1β (interleukin-1 beta); elevated TNF-α (tumor necrosis factor alpha); elevated Iba1; elevated GFAP; depressed NeuN; depressed TGF-beta (Transforming growth factor beta); elevated Interferon-γ (IFN-γ); and
elevated inducible Nitric Oxide Synthase (iNOS).

6. The method of claim 5, wherein the Cognitive Deficit (CD) subscale of the SCL-90-R Scale comprises the symptoms of
headaches; trouble remembering; temper outburst; do things slow; double check;
mind goes blank; trouble remembering; and wrong with body.

7. The method according to claim 1, wherein the method reduces, slows, reverses or prevents an increase of the Cognitive Deficit (CD) subscale of the SCL-90-R Scale in the subject.

8. The method according to claim 1, wherein the tdsRNA modulates an immune system in the subject by binding a Toll-like receptor 3 (TLR3) receptor in the subject.

9. The method according to claim 1, wherein the tdsRNA crosses the blood-brain barrier in the subject.

10. The method of claim 1, wherein the method reduces, stops or reverses in the subject at least one selected from the group consisting of:
a neuroinflammatory disorder symptom;
a neuroinflammatory disorder pathology; and
a neuroinflammatory disorder sign.

11. The method of claim 1, wherein n is a number with a value which is at least one selected from the group consisting of:
40 to 50,000; 40 to 40,000; 50 to 10,000; 60 to 9000; 70 to 8000; 80 to 7000;
and 380 to 450.

12. The method of claim 1,
wherein n is from 40 to 40,000;
wherein the tdsRNA has about 4 to about 4000 helical turns of duplexed RNA strands; or
wherein the tdsRNA has a molecular weight selected from the group consisting of:
2 kDa to 30,000 kDa;
25 kDa to 2500 kDa; and
250 kDa to 320 kDa.

13. The method of claim 1, wherein the tdsRNA comprises
rIₙ·ribo(C₁₁₋₁₄U)ₙ; and
rugged dsRNA.

14. The method of claim 1, wherein the rugged dsRNA has
a single strand comprised of r(C₄₋₂₉U)ₙ, r(C₁₁₋₁₄U)ₙ, or r(C₁₂U)ₙ; and
an opposite strand comprised of r(I);
wherein the single strand and the opposite strand do not base pair the position of the uracil base; and
wherein the single strand and the opposite strand are partially hybridized.

15. The method of claim 1,
wherein
the rugged dsRNA has a molecular weight of about 250 kDa to 500 kDa;
each strand of the rugged dsRNA is from about 400 to 800 basepairs in length;
or
the rugged tdsRNA has about 30 to 100 or 30-60 helical turns of duplexed RNA.

16. The method of claim 1, wherein the tdsRNA is Rugged dsRNA which is resistant to denaturation under conditions that are able to separate hybridized poly(riboinosinic acid) and poly(ribocytosinic acid) strands (rIₙ•rCₙ).

17. The method of claim 1, wherein the rugged dsRNA is an isolated double-stranded ribonucleic acid (dsRNA) enzymatically active under thermal stress comprising:
each strand with a molecular weight of about 250 KDa to about 500 KDa, 400-800 basepairs, or 30 to 60 helical turns of duplex RNA;
a single strand comprised of poly(ribocytosinic₄₋₂₉ uracilic acid) and an opposite strand comprised of poly(riboinosinic acid);
wherein the two strands do not base pair the position of the uracil base;
wherein the two strands base pair the position of the cytosine base; and
wherein said strands are partially hybridized.

18. The method of claim 1, wherein the composition comprises at least one pharmaceutically acceptable carrier.

19. The method of claim 1, wherein administering is at least one administering method selected from the group consisting of:
systemic administration; intravenous administration; intradermal administration; subcutaneous administration; intramuscular administration; intranasal administration; pulmonary airway administration; intranasal administration and oral administration; intraperitoneal administration; intracranial administration; intravesical administration; oral administration; topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration;
lung administration; nasal passage administration; respirable solid administration;
respirable liquid administration; and dry powder inhalants administration.

20. The method of claim 1, wherein the tdsRNA is administered at a dosage of about
25 mg to 700 mg of tdsRNA per day; 20 mg to 200 mg of tdsRNA per day; 50 mg to 150 mg of tdsRNA per day; or 80 mg to 140 mg of tdsRNA per day.

21. The method of claim 1, wherein the tdsRNA is administered at a rate selected from the group consisting of:
one dose per day; one dose every 2 days; one dose every 3 days; one dose every 4 days; one dose every 5 days; one dose a week; two doses a week; three doses a week;
one dose every two weeks; one dose every 3 weeks; one dose every 4 weeks;
and one dose a month.

22. A composition for treating reducing, or preventing neuroinflammation in a subject comprising at least one selected from the group consisting of: 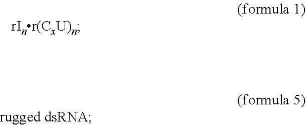
wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

23. The composition of claim 22, wherein the neuroinflammation is at least one selected from the group consisting of a neuroinflammation disorder; a neuroinflammation disorder symptom; a neuroinflammation disorder pathology; a neuroinflammation disorder sign; neuroinflammation; depression; schizophrenia; Alzheimer's disease (AD); Parkinson's disease; Multiple Sclerosis (MS); postoperative cognitive dysfunction (POCD); spinal cord injury (SCI); AIDS dementia complex (ADC); ischemia; stroke; traumatic brain injury (TBI); infection of the brain or central nervous system; brain tumors; frontotemporal dementia; amyotrophic lateral sclerosis (ALS); multi-system atrophy; Acute disseminated encephalomyelitis (ADEM); Acute Optic Neuritis (AON); Transverse Myelitis; and Neuromyelitis Optica (NMO).

24. The composition of claim 22, wherein the disorder symptom, disorder pathology, or disorder sign is at least one selected from the group consisting of
or the is at least one selected from the group consisting of:
amyloid accumulation; neurofibrillary tangles; cognitive function decline; beta-amyloid accumulation; neurofibrillary tangles accumulation; neuroinflammation; tau protein level in cerebrospinal fluid; beta-amyloid level in cerebrospinal fluid; a high or increasing score on a Cognitive Deficit (CD) subscale of the SCL-90-R scale; microglial activation; astrocyte activation; elevated IL-6 (interleukin-6); elevated IL-23 (interleukin-23); elevated IL-1β (interleukin-1 beta); elevated TNF-α (tumor necrosis factor alpha); elevated Iba1; elevated GFAP; depressed NeuN; depressed TGF-beta (Transforming growth factor beta); elevated Interferon-γ (IFN-γ); and
elevated inducible Nitric Oxide Synthase (iNOS).
